(19) United States
(12) Patent Application Publication
(10) Pub. No.: US 2013/0211353 A1
(43) Pub. Date: Aug. 15, 2013

(54) PERCUTANEOUS ABSORPTION TYPE FORMULATION

(71) Applicants: Arata Toshimitsu, Tsukuba-shi (JP); Hisakazu Kurita, Tsukuba-shi (JP)

(72) Inventors: Arata Toshimitsu, Tsukuba-shi (JP); Hisakazu Kurita, Tsukuba-shi (JP)

(73) Assignee: Hisamitsu Pharmaceutical Co., Inc., Tosu-shi, Saga (JP)

(21) Appl. No.: 13/881,829

(22) PCT Filed: Oct. 26, 2011
(86) PCT No.: PCT/JP2011/074687

§ 371 Date: Apr. 26, 2013

(30) Foreign Application Priority Data
Oct. 28, 2010 (JP) 2010-242252

(51) Int. Cl. A61K 47/18; A61K 47/14; A61M 35/00

(52) U.S. Cl. CPC A61K 47/18; A61M 35/00; A61K 47/14
USPC 604/307; 514/786; 514/399; 514/415

(57) ABSTRACT

Provided is a percutaneous absorption type formulation of a drug such as imidafenacin and silodosin, wherein stable absorption through the skin is realized. The percutaneous absorption type formulation containing a drug such as imidafenacin and silodosin further contains a transdermal absorption promoting agent, and fatty acid ester and/or fatty acid amide that further improve(s) the function of the transdermal absorption promoting agent.

### TECHNICAL FIELD

The present invention relates to a percutaneous absorption preparation comprising a percutaneous absorption promoting agent, characterized in that it further comprises a fatty acid ester and/or a fatty acid amide that further improve the function of the percutaneous absorption promoting agent.

### BACKGROUND ART

In order to obtain drug efficacy by administering a drug, an oral administration method is generally used; however, transdermal administration method has many advantages compared to oral administration method. For example, in oral administration method, a drug absorbed in the bowel is first metabolized in the liver so that a large amount of the drug is decomposed before it exhibits drug efficacy in desired regions; whereas in transdermal administration method, the absorbed drug does not initially pass through the liver in the body circulation, and therefore its efficacy is not significantly decreased in the liver due to metabolism. Furthermore, transdermal administration method has other advantages such as, that the drug effect is continuous, and it has a sustained drug release characteristics.

In addition, as an advantage of transdermal administration method, reduction of side effects can be expected by sustained release of drugs and by maintaining their constant blood levels. In particular, there is a tendency that transdermal administration preparations that can be administered for a long period of time (1 day to 7 days) are desired from the viewpoint of patient compliance.

In such percutaneous absorption preparations, an important issue is how to effectively release the drug (medicinal ingredient) from a base, namely, to effectively transfer the drug from the base to the skin. In general, when formulation design is attempted using a specific drug, quite often crystallization, etc. occurs due to insufficient dissolution of the drug in a base, resulting in a decreased level of drug release and insufficient therapeutic effect. Furthermore, because a drug is absorbed through the skin, it is necessary to increase the skin permeability of the drug. Therefore, selection of the optimal dissolving agent for the drug is important in the formulation design; depending on the selection of dissolving agent, dissolution of the drug becomes insufficient, leading to a decreased level of release of the drug from the base and decreased level of transfer of the drug to a diseased part, and consequently, sufficient therapeutic effect cannot be exerted.

Imidafenacin

(4-(2-methyl-1H-imidazol-1-yl)-2-2-diphenylbutane amide) is a muscarinic receptor antagonist having a M3 and M1 muscarinic receptor antagonistic activity selective for the bladder, and is a therapeutic drug for urinary frequency and urinary incontinence.

Silodosin is a selective α₁-blocker that selectively acts on the prostate and urethra, and is a therapeutic drug to improve dysuria.

While imidafenacin and silodosin have been used as an agent for oral administration in the current clinical setting, from the viewpoints of reduction of side effects such as liver failure, stabilization of blood concentration for a long period of time, and long-sustaining effects, development of transdermal administration preparations such as adhesive patch, etc., rather than oral administration preparations, has been desired.

Based on such current situation, percutaneous absorption preparations comprising imidafenacin and/or silodosin have been proposed (Patent Literatures 1 to 3).

In Patent Literatures 1 and 2, a percutaneous absorption preparation comprising 4-(2-methyl-1H-imidazo-1-yl)-2,2-diphenylbuntane amide (imidafenacin) is described, and in Patent Literature 3, a percutaneous absorption preparation comprising silodosin is described.

However, since imidafenacin and silodosin have low skin permeability, in order to use them in a skin-absorption type preparation wherein a drug is absorbed through the skin, their skin permeability must be increased. However, in Patent Literatures 1 to 3, a means for solving such a problem has not been provided.

### CITATION

### Patent Literature

Patent Literature 1: WO 2005/011683

Patent Literature 2: WO 2006/082888

Patent Literature 3: WO 03/024432

### SUMMARY OF INVENTION

### Technical Problem

Therefore, the present inventors have obtained recognition that regarding percutaneous absorption preparations comprising a drug such as imidafenacin and silodosin, by increasing the skin permeability of the drug, a skin-absorption type preparation that enables stable absorption of the drug through the skin should be produced. Namely, the problem to be solved by the present invention is to provide a percutaneous absorption preparation that achieves stable absorption of a drug such as imidafenacin and silodosin through the skin.

### Solution to Problem

Through extensive research to solve the above problem, the present inventors have discovered that, in a percutaneous absorption preparation comprising a drug such as imidafenacin and silodosin, stable absorption of the drug can be achieved by comprising a percutaneous absorption promoting agent, and by further comprising fatty acid ester and/or fatty acid amide that further improve the function of the percutaneous absorption promoting agent; and the present inventors have accomplished the present invention.

Namely, the present invention relates to the followings:

(a) A percutaneous absorption preparation comprising a drug and one or more percutaneous absorption promoting agent(s) selected from triacetin, isopropyl myristate, oleyl alcohol, octyldodecanol and stearyl alcohol for said drug, wherein the percutaneous absorption preparation further comprises a fatty acid ester and/or a fatty acid amide that further improve the function of the percutaneous absorption promoting agents.
(b) The percutaneous absorption preparation according to (a), wherein the drug is imidafenacin and/or a salt thereof, or silodosin and/or a salt thereof.
(c) The percutaneous absorption preparation according to (a) or (b), wherein the fatty acid ester is sorbitan monolaurate.
(d) The percutaneous absorption preparation according to any one of (a) to (c), wherein the fatty acid amide is lauric acid diethanolamide.
(e) The percutaneous absorption preparation according to any one of (a) to (d), which is a skin external patch that comprises a pressure-sensitive adhesive composition comprising:
(1) imidafenacin and/or a salt thereof, or silodosin and/or a salt thereof, as a drug,
(2) triacetin,
(3) sorbitan monolaurate and/or lauric acid diethanolamide,
(4) a pressure-sensitive adhesive base.
(f) The percutaneous absorption preparation according to any one of (a) to (c), which is a skin external patch that comprises a pressure-sensitive adhesive composition comprising:
(1) imidafenacin and/or a salt thereof as a drug,
(2) triacetin, isopropyl myristate, oleyl alcohol, octyldodecanol, or stearyl alcohol,
(3) sorbitan monolaurate,
(4) a pressure-sensitive adhesive base.
(g) The percutaneous absorption preparation according to (f), wherein at 15 hr after its application, the skin permeation rate is greater than 2.0 μg/cm²/hr and the cumulative amount of permeation is greater than 30 μg/cm².
(h) The percutaneous absorption preparation according to (e), wherein it comprises silodosin and/or a salt thereof as a drug, and at 13 hr after its application, the skin permeation rate is greater than 7.0 μg/cm²/hr and the cumulative amount of permeation is greater than 100 μg/cm².
(i) The percutaneous absorption preparation according to any one of (e) to (h), wherein the pressure-sensitive adhesive base is (meth)acrylic acid ester copolymer.
(j) The percutaneous absorption preparation according to any one of (e) to (i), which is a skin external patch having a structure in which the pressure-sensitive adhesive composition is laminated on a backing and covered with a liner.
(k) A method for producing a percutaneous absorption preparation comprising a drug and one or more percutaneous absorption promoting agents selected from triacetin, isopropyl myristate, oleyl alcohol, octyldodecanol and stearyl alcohol for said drug, wherein the percutaneous absorption preparation has increased skin permeability by further comprising a fatty acid ester and/or a fatty acid amide that further improve the function of said percutaneous absorption promoting agents.

### Advantageous Effects of Invention

The percutaneous absorption preparation of the present invention enables stable absorption of a drug such as imidafenacin and silodosin through the skin, so that it is extremely useful for the treatment of urinary frequency, urinary incontinence and dysuria.

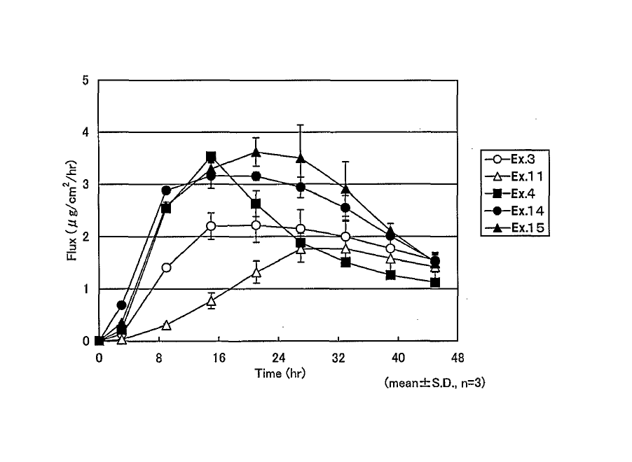

### BRIEF DESCRIPTION OF DRAWINGS

[FIG. 1] FIG. 1 is a graph showing time-course change in the amount of permeation.

### DESCRIPTION OF EMBODIMENTS

The percutaneous absorption preparation of the present invention is a percutaneous absorption preparation that comprises a drug, as well as triacetin, isopropyl myristate, oleyl alcohol, octyldodecanol or stearyl alcohol as a percutaneous absorption promoting agent for said drug, and that further comprises a fatty acid ester and/or a fatty acid amide which further improve the function of the percutaneous absorption promoting agent.

As the percutaneous absorption promoting agent that can be used in the percutaneous absorption preparation of the present invention, any compound that has conventionally been recognized to have an absorption promoting action in the skin may be used, and examples include, fatty acids having 6-20 carbon chains, fatty alcohols, fatty acid esters, fatty acid amides, or fatty acid ethers, aromatic organic acids, aromatic alcohols, aromatic organic acid esters or ethers (the above may be either saturated, or unsaturated, and any of cyclic, straight-chain and branched, chain), and furthermore, lactic acid esters, acetic acid esters, monoterpene compounds, sesquiterpene compounds, Azone, Azone derivatives, pyrothiodecane, glycerin fatty acid esters, propylene glycol fatty acid esters, sorbitan fatty acid esters (Span series) polysorbates (Tween series), polyethylene glycol fatty acid esters, polyoxyethylene hardened castor oils type (HCO series), polyoxyethylene alkyl ethers, sucrose fatty acid esters, and vegetable oils, etc.; the percutaneous absorption promoting agent can be appropriately selected depending on the intended use (dose regimen and dosage).

Preferred percutaneous absorption promoting agents are triacetin, isopropylmyristate, oleyl alcohol, octyldodecanol and stearyl alcohol; such percutaneous absorption promoting agents can synergistically improve the skin permeability of imidafenacin and silodosin by their concomitant use with fatty acid esters and fatty acid amides.

Two or more kinds of percutaneous absorption promoting agents may be mixed and co-used; with consideration given to sufficient permeability as a percutaneous absorption preparation and skin irritation such as redness and edema, etc., the percutaneous absorption promoting agents may be blended in an amount of 0.01-40 mass %, more preferably 0.05-30 mass %, and particularly preferably 0.1-20 mass %, based on the weight of the total composition of the percutaneous absorption preparation.

The blending ratio of triacetin to sorbitan monolaurate in the percutaneous absorption preparation of the present invention is not particularly limited, and is preferably 1:1 to 6:1, more preferably 3:1 to 6:1.

The blending ratio of triacetin to lauric acid diethanolamide in the percutaneous absorption preparation of the present invention is not particularly limited, and is preferably 1:1 to 9:1, more preferably 1:1 to 5:1, and most preferably 1:1 to 3:1.

The blending ratio of isopropyl myristate as a percutaneous absorption promoting agent relative to sorbitan monolaurate in the percutaneous absorption preparation of the present invention is not particularly limited, and is preferably 1:5 to 5:1, more preferably 1:3 to 3:1, and most preferably 1:2 to 2:1.

The blending ratio of oleyl alcohol, octyldodecanol or stearyl alcohol as a percutaneous absorption promoting agent relative to sorbitan monolaurate or lauric acid diethanolamide in the percutaneous absorption preparation of the present invention is not particularly limited, and is preferably 1:5 to 5:1, more preferably 1:3 to 3:1, and most preferably 1:2 to 2:1.

The amount of imidafenacin contained in the percutaneous absorption preparation of the present invention is preferably 0.5-10 mass %, and more preferably 1-5 mass %.

The amount of silodosin contained in the percutaneous absorption preparation of the present invention is preferably 1-10 mass %, and more preferably 3-7 mass %.

The percutaneous absorption preparation of the present invention may comprise isostearic acid as a crystallization inhibitor.

There is no particular limitation on the dosage form of the percutaneous absorption preparation of the present invention, and dosage forms conventionally used as a skin external preparation may be used; for example, any dosage forms including skin external patch, poultice, plaster, ointment, gels, creams, lotion, reservoir-type patch, liniment, aerosol and others may be used for the percutaneous absorption preparation.

The skin external patch has preferably a structure in which a pressure-sensitive adhesive composition is laminated on a backing and covered with a liner.

As a backing of skin external patch, the following materials with and without stretching properties are selected: for example, a film, a sheet or a foil of polyethylene, polypropylene, polybutadiene, ethylene-vinyl acetate copolymer, polyvinyl chloride, polyesters such as polyethylene terephthalate (PET), polybutyleneterephthalate and polyethylene naphthalate, nylon, polyurethane, cotton, rayon (cellulose derivatives), and aluminum, etc., and a porous form and a foam thereof, as well as paper, woven fabric, knitted fabric, and nonwoven fabric, etc., and laminates thereof can also be used.

As a liner of skin external patch, a film, a sheet, or a foil of polyethylene, polypropylene, polyesters such as polyethylene terephthalate (PET) and polyethylene naphthaiate, nylon, aluminum, etc., or paper, etc. may be selected; and a laminate thereof may also be used. In addition, in order to facilitate peeling of the pressure-sensitive adhesive, the surface of said liner may be treated with silicone. Teflon (registered trademark), a surfactant, etc.

Next, poultice and plaster will be explained. For example, as a base of poultice, with consideration given to time-course stability, release characteristics, percutaneous absorption characteristics and safety for the skin, a hydrophilic base blending water-soluble polymer, polyhydric alcohol and water is used.

As the water-soluble polymer used in this hydrophilic base, one or trio re kinds are appropriately selected from the following: gelatin, casein, pullulan, dextran, sodium alginate, soluble starch, carboxymethyl starch, dextrin, carboxymethyl cellulose, carboxymethyl cellulose sodium, methyl cellulose, ethyl cellulose, hydroxyethyl cellulose, polyvinyl alcohol, polyethylene oxide, polyacrylic acid, polyacrylamide, sodium polyacrylate, polyvinyl pyrrolidine, carboxyvinyl polymer, polyvinyl ether, methoxy ethylene-maleic anhydride copolymer, isobutylene-maleic anhydride copolymer, N-vinyl acetamide, copolymer of N-vinyl acetamide and acrylic acid and/or acrylate. In this case, the amount of water-soluble polymer blended is 1-30 mass %, preferably 1-20 mass %, and more preferably 1-15 mass % relative to the total amount of the preparation. When the amount of blending is too small, viscosity decreases and the degree of shape retention decreases; when the amount of blending is too large, viscosity increases and workability during kneading and coating decreases.

As the polyhydric alcohol, one kind, or two or more kinds as necessary, are appropriately selected from the following: polyethylene glycol, propylene glycol, dipropylene glycol, polypropylene glycol, 1,3-butylene glycol, 1,4-butylene glycol, isobutylene glycol, glycerin, diglycerin, sorbitol, etc.; and its amount of blending is 10-90 mass %, preferably 10-70 mass %, and more preferably 20-60 mass %. When the amount of blending is too small, moisture-retaining effect decreases; when it is too large, solubility of water-soluble polymer is affected. The amount of water blended is 10-90 mass %, preferably 20-80 mass %; water is necessary to dissolve water-soluble polymers and to exhibit their viscosity, cohesiveness and shape retention characteristic.

Furthermore, in addition to the above essential components, one or more crosslinking agents may be appropriately blended as necessary, and examples of the crosslinking agents include polyvalent metal compounds, and specifically, aluminum hydroxide, aluminum chloride, calcium hydroxide, calcium chloride, aluminum sulfate, aluminum ammonium sulfate, aluminum potassium sulfate, magnesium aluminometasilicate, dihydroxy aluminum aminoacetate, etc.; other examples of the crosslinking agents include compounds having at least two epoxy groups in the molecule, and specifically, ethylene glycol diglycidyl ether, polyethylene glycol diglycidyl ether, propylene glycol diglycidyl ether, polypropylene glycol diglycidyl ether, polytetramethylene glycol diglycidyl ether, glycerol polyglycidyl ether, polyglycerol polyglycidyl ether, sorbitol polyglycidyl ether, sorbitan polyglycidyl ether, trimethylol propane polyglycidyl ether, pentaerythritol polyglycidyl ether, resorcinol diglycidyl ether, neopentyl glycol diglycidyl ether, 1,6-hexanediol diglycidyl ether, etc.

In addition, one or more components consisting of a filler such as kaolin, zinc oxide, titanium dioxide, talc, bentonite, synthetic aluminum silicate, etc., a preservative such as thymol, methyl paraben, ethyl paraben, etc., an antioxidant such as ascorbic acid, stearic acid ester, dibutyl hydroxy toluene, butyl hydroxy anisole, gallic acid ester, vitamin E, vitamin E acetate, disodium edetate, etc., a UV absorber such as 2-hydroxy-4-methoxybenzophenone, ethyl p-aminobenzoate, 2-(2-hydroxy-5-methylphenyl) benzotriazole, glycol salicylate, methyl salicylate, phenyl salicylate, etc., and an emulsifying agent such as sorbitan fatty acid ester, glycerin fatty acid ester, decaglycerol fatty acid ester, polyoxyethylene sorbitan fatty acid ester, polyethylene glycol fatty acid ester, polyoxyethylene alkyl ether, etc. may be appropriately blended.

As a backing of this poultice, it is important to select a material that does not affect the release of medicinal ingredients. Namely, a backing having no interaction with and no adsorption of medicinal ingredients is important. For example, a backing is selected from a film or a sheet of polyethylene, polypropylene, polyvinyl chloride, polyester, nylon, polyurethane, etc., or a porous form and a foam thereof, as well as fabric and nonwoven fabric; or it may be selected from a laminate of the film or sheet with the porous body, foamed body, fabric, or nonwoven fabric. Furthermore, as a covered material for peeling, polyethylene, polypropylene, polyester, or these materials mold-release-treated with silicone, as well as release paper, etc. may be used.

Next, methods for producing these poultices will be described; poultices are what can be easily produced by an already known method for producing. For example, a water-soluble polymer is mixed into polyhydric alcohol and water, dispersed and dissolved to make a homogenous kneaded mixture, to which a stabilizer, an antioxidant, an UV absorber, an emulsifier, a preservative, and an antibacterial agent are added as necessary. Then, medicinal ingredients are added, homogeneously dispersed, and the resulting mixture is directly spread on a backing; or it is once spread on a paper or film that has been mold-release-treated, then pressure-transferred onto the backing used. Here, the sequence of blending each base, medicinal ingredients as well as other components in the above production method is described only as an example, and the blending sequence is not limited thereto.

Next, regarding the plaster, its pressure-sensitive adhesive base may be appropriately selected from those known in the art in consideration of safety for the skin, release characteristics of medicinal ingredients, and adhesiveness to the skin, etc. Preferred pressure-sensitive adhesive includes acrylic type pressure-sensitive adhesive, rubber type pressure-sensitive adhesive, silicone type pressure-sensitive adhesive, etc.

The acrylic pressure-sensitive adhesive is not particularly limited as long as it is a copolymer comprising at least one (meth)acrylic acid derivative represented by 2-ethylhexyl acrylate, methyl acrylate, butyl acrylate, hydroxyethyl acrylate, 2-ethylhexyl methacrylate, etc. Examples that can be used include pressure-sensitive adhesives listed in “2007 Encyclopedia of Pharmaceutical Excipients” (edited by Japan Pharmaceutical Excipients Council), such as acrylic acid-acrylic acid octyl ester copolymer, 2-ethylhexyl acrylate/vinylpyrrolidone copolymer solution, acrylic acid ester-vinyl acetate copolymer, 2-ethylhexyl acrylate/2-ethylhexyl methacrylate/dodecyl methacrylate copolymer, methyl acrylate/2-ethylhexyl acrylate copolymer resin emulsion, and acrylic polymers contained in an acrylic resin alkanolamine solution, as well as Eudragit, etc. (Higuchi Shokai Co., Ltd.), DURO-TAK acrylic pressure-sensitive adhesive series (from Henkel). In particular, acrylic pressure-sensitive adhesives having a hydroxyl group can be preferably used from the viewpoint of drug release characteristics.

Examples of the rubber pressure-sensitive adhesive include natural rubber, polyisoprene rubber, polyisobutylene, polyvinyl ether, polyurethane, polyisoprene, polybutadiene, styrene-butadiene copolymer, styrene-isoprene copolymer, styrene-isoprene-styrene block copolymer, etc. As the silicone pressure-sensitive adhesive, those comprising polyorganosiloxane and polydimethyl siloxane as main ingredients are used.

As tackifiers which can be used in this setting, rosin types such as rosin, and hydrogenated, disproportionated, polymerized, and esterified rosin derivatives; terpene resin such as α-pinene, β-pinene, etc.; terpene-phenol resin, fatty acid-, aromatic-, alicyclic-, copolymerized-petroleum resins, as well as alkyl-phenyl resin, xylene resin, etc. can be exemplified.

A softening agent is an agent that plasticizes and softens base polymers to retain their adequate adhesion to the skin. Examples of such softening agent include polybutene, poly isobutylene, liquid paraffin, higher fatty acid esters such as isopropylmyristate, etc, and silicon oil, vegetable oils such as almond oil, olive oil, camellia oil, persic oil, and peanut oil.

As a backing of plaster, those which do not affect the release of medicinal ingredients are preferred, and those having stretching properties or non-stretching properties are used. For example, the backing is selected from a film or a sheet made from synthetic resins such as polyethylene, polypropylene, polybutadiene, ethylene-vinyl acetate copolymer, polyvinyl chloride, polyester, nylon, polyurethane, etc., and a laminate, a porous film, and a foam thereof, as well as paper, fabric and non-woven fabric, etc.

This plaster can be easily produced by a conventionally known method for producing; for example, in the case of synthetic rubber tape, a pressure-sensitive adhesive base, a softening agent and a tackifier are mixed by heating at 120-160° C. using a mixing machine such as kneader or mixer, etc., and a medicinal ingredient is added to the mixture, then the mixture is directly spread on a polypropylene or polyester film, etc.; alternatively, the mixture is once spread on a sheet or film that has been mold-release-treated, which then covers a desired backing and is pressure-transferred on the backing.

In the case of plasters that use an acrylic pressure-sensitive adhesive, a pressure-sensitive adhesive base, a medicinal ingredient and an absorption promoting agent, and an additive as necessary are dissolved or dispersed in an appropriate solvent, and the resulting solution or fluid dispersion is directly applied on the surface of a backing, dried, to form typically an adhesive layer with a thickness of 30-200 μm. Alternatively, this solution or dispersion may be applied on a protective releasing paper, then the resulting adhesive layer obtained after drying may be attached to a backing. The solvent used in this method for producing, which is not particularly limited as long as it is an organic solvent having compatibility with all the blending components such as pressure-sensitive adhesive base and medicinal ingredients, etc., includes aromatic hydrocarbons such as toluene, benzene and xylene, etc., esters such as ethyl acetate, etc., halogenated hydrocarbons such as carbon tetrachloride, chloroform and methylene chloride, etc.

The base polymer of this plaster can be appropriately selected from those known in the art in consideration of safety for the skin, release characteristics of medicinal ingredients and adhesion to the skin, etc.; styrene-isoprene-styrene block copolymer having extremely low polarity is preferred. In addition, while a styrene-isoprene-styrene block copolymer is preferably exemplified above as a base polymer, it may be co-used with other polymers, such as polyisobutylene, etc.

A softening agent plasticizes and softens styrene-isoprene-styrene block copolymer, i.e., a base polymer, to maintain its adequate adhesiveness to the skin. As the softening agent, almond, oil, olive oil, camellia oil, persic oil, peanut oil, liquid paraffin, etc. are used. Its blending ratio is preferably 150-350 parts by weight relative to 100 parts by weight of the styrene-isoprene-styrene block copolymer, from the viewpoint of ensuring sufficient level of viscosity.

Next, the blending recipe for other types of percutaneous absorption preparations, such as ointment, gels, creams, gelled creams, lotion, reservoir-type patch, liniment, and aerosol is briefly explained.

An ointment comprises, in addition to medicinal ingredients, at least a higher fatty acid such as myristic acid or an ester thereof, waxes such as spermaceti, etc., a surfactant such as polyoxyethylene, and hydrocarbons such as hydrophilic petrolatum, etc.

In the formulation of this ointment, for example, 5-15 mass % of a higher fatty acid or an ester thereof, 1-10 mass % of a surfactant, and 0.5-10 mass % of a medicinal ingredient are mixed at room temperature or under heating, then 4-10 mass % of a wax and 50-90 mass % of a hydrocarbon are added and either heated or melted by heating, kept at 50-100° C.; after all the components become a clear solution, it is homogeneously mixed by a homomixer. Then, it is cooled to room temperature with stirring to obtain an ointment.

Gels comprise, in addition to medicinal ingredients, at least a lower alcohol such as ethanol, water, a gelling agent such as carboxyvinyl polymer, and a neutralizing agent such as triethanolamine, etc.

In the formulation of the gels, for example, 0.5-5 mass % of a gelling agent is added to 55 mass % or less of water and allowed to swell. Meanwhile, 0.5-10 mass % of a medicinal ingredient is dissolved in a mixture of 40 mass % or less of a glycol with 60 mass % or less of a lower alcohol. These two mixtures are mixed, to which a neutralizing agent is added to adjust the pH to 4-7, and the gels are obtained.

Creams comprise, in addition to medicinal ingredients, at least a higher fatty acid ester such as myristic acid ester, water, hydrocarbons such as liquid paraffin, an emulsifying agent such as polyoxyethylene alkyl ether.

In the formulation of the creams, they are obtained by adding appropriate amounts of the above medicinal ingredient, higher fatty acid ester, hydrocarbon, and emulsifying agent, and by mixing and stirring the mixture.

Gel-cream has properties intermediate between gels and creams; it is obtained by blending, in addition to the above each component of the creams, a gelling agent such as carboxyvinyl polymer, etc., and a neutralizing agent such as diisopropanolamine, then by adjusting the pH to 4-8, preferably 5-6.5.

In the formulation of this gel-cream, for example, 0.5-10 mass % of a medicinal ingredient is dissolved in a mixture of 25 mass % or less of a higher fatty acid ester and 40 mass % or less of a lower alcohol, to which 5 mass % or less of an emulsifying agent is added. Meanwhile, 0.5-5 mass % of a gelling agent is added to water and allowed to swell. Then, these two mixtures are mixed using a homomixer and homogeneously emulsified, after which a neutralizing agent is added to adjust the pH to 4-8.

A lotion comprises, in addition to medicinal ingredients, at least a lower alcohol such as ethanol, etc., water and/or glycols.

In the formulation of this lotion, it is obtained by adding appropriate amounts of the above medicinal ingredient, lower alcohol, water and/or glycols, and by mixing and stirring them.

A reservoir-type patch is composed at least of (1) a backing layer, (2) a drug reservoir layer, (3) a drug release layer, and (4) a pressure-sensitive adhesive layer, wherein said (2) drug reservoir layer is composed of, in addition to medicinal ingredients, a base that comprises either (a) at least glycols, lower alcohol, water-soluble polymer, (b) at least aliphatic alcohol and polyhydric alcohol, or (c) at least paraffin and silicon.

A liniment comprises, in addition to medicinal ingredients, at least an alcohol such as ethanol and polyethylene glycol, water, a fatty acid ester such as adipic acid and sebacic acid.

In the formulation of the liniment, it is obtained by mixing and stirring 0.5-10 mass % of a medicinal ingredient with 10-70 mass % of an alcohol, 55 mass % or less of water, and 60 mass % or less of a fatty acid ester.

An aerosol comprises, in addition to medicinal ingredients, at least a lower alcohol, water, dimethyl ether and/or liquefied petroleum gas; and medicinal adjuvants such as camphor, α-tocopherol, menthol, etc., may be blended as desired.

In the specific formulation of the aerosol, 0.5-10 mass % of a medicinal ingredient is blended with lower alcohol and water, filled into an aerosol container, to which dimethyl ether and/or liquefied petroleum gas as a propellant are pressure-injected; and the aerosol is obtained.

Within the range that does not impair the object of the present invention, various pharmaceutically acceptable additives, such as stabilizers, antioxidants, perfumes, fillers, UV absorbers, preservatives, antimicrobial agents, and other percutaneous absorption promoting agents can be added.

### EXAMPLES

Hereinafter, the present invention is described in further detail with reference to examples; however, the present invention is not limited to these examples. Unless stated otherwise, “%” represents “mass %”.

TABLE 1
[Evaluation of skin permeability of imidafenacin (triacetin)]

| | | | | | Skin permeability in | | |
|---|---|---|---|---|---|---|---|
| | | | | | hairless mouse | | |
| | | | | | | | Cumulative |
| | | | | Sorbitan | | | amount of |
| | | Imidafenacin | Triacetin | monolaurate | Jₘₐₓ | Tₘₐₓ | permeation |
| | | (mass %) | (mass %) | (mass %) | (μg/cm²/hr) | (hr) | (μg/cm²) |
| Com. | Ex. 1 | 1.5 | — | — | 0.47 | 33 | 3.0 |
| Ex. | Ex. 2 | 1.5 | 10 | — | 0.44 | >46 | 3.5 |
| | Ex. 3 | 1.5 | — | 5 | 2.22 | 21 | 35.8 |
| Ex. | Ex. 4 | 1.5 | 10 | 5 | 4.30 | 15 | 68.2 |
| | Ex. 5 | 1.5 | 5 | 5 | 3.53 | 15 | 53.5 |
| | Ex. 6 | 1.5 | 7 | 5 | 4.06 | 15 | 61.7 |
| | Ex. 7 | 1.5 | 10 | 3 | 3.23 | 21 | 42.7 |
| | Ex. 8 | 1.3 | 7 | 5 | 2.96 | 21 | 39.0 |
| | Ex. 9 | 1.7 | 7 | 5 | 4.48 | 15 | 71.6 |

(Jₘₐₓ: Maximum skin permeation rate, Tₘₐₓ: Time to reach the maximum skin permeation rate)

Imidafenacin, triacetin and sorbitan monolaurate were comprised in an OH-group-containing acrylic pressure-sensitive adhesive base so as to achieve the blending ratios described in the above Table, and percutaneous absorption preparations were prepared. Permeability of each of the percutaneous absorption preparations through the skin of a hairless mouse was measured as follows.

On the side of the stratum corneum of the skin (at lateral side of the body) removed from a hairless mouse, the above percutaneous absorption preparation was adhered, i.e., the above percutaneous absorption preparation was applied, and the skin was mounted on a flow-through type diffusion cell with the dermis side placed at the receptor phase side. At the receptor phase, phosphate buffered saline of pH 7.4 was circulated to maintain the skin surface temperature at 32±1° C., and samples were collected at regular intervals, drug concentrations were measured by high performance liquid chromatography, and skin permeation rates (Flux (μg/cm²/hr)) were calculated.

Regarding the pressure-sensitive adhesive compositions as comparative examples, i.e., that without triacetin and sorbitan monolaurate (Example 1), and that with any one of them (Examples 2 and 3), a long time was required to reach the maximum skin permeation rate, and the value of the maximum skin permeation rate was low. In contrast, regarding the pressure-sensitive adhesive compositions as examples containing both triacetin and sorbitan monolaurate (Examples 4-9), a skin permeation rate of greater than 2.0 μg/cm²/hr was measured at 15 hr after the application; namely, a high Jₘₐₓ value could be obtained within a short period of time (Tₘₐₓ), and also a large cumulative amount of permeation exceeding 30 μg/cm² was obtained.

Thus, the percutaneous absorption preparations comprising both triacetin and sorbitan monolaurate were demonstrated to have synergistically increased skin permeability of imidafenacin.

TABLE 2
[Evaluation of skin permeability of imidafenacin (sorbitan
monolaurate)]

| | | | | | | Skin permeability in | | |
|---|---|---|---|---|---|---|---|---|
| | | | | Percutaneous | | hairless mouse | | |
| | | | | absorption | | | | Cumulative |
| | | | Sorbitan | promoting | | | | amount of |
| | | Imidafenacin | monolaurate | agent | | Jₘₐₓ | Tₘₐₓ | permeation |
| | | (mass %) | (mass %) | (mass %) | | μg/cm²/hr | (hr) | (μg/cm²) |
| Com. | Ex. 1 | 1.5 | — | — | — | 0.47 | 33 | 3.0 |
| Ex. | Ex. 3 | 1.5 | 5 | — | — | 2.22 | 21 | 35.8 |
| | Ex. 10 | 1.5 | — | Isopropyl | 10 | 1.32 | 33 | 14.8 |
| | | | | myristate | | | | |
| | Ex. 11 | 1.5 | — | Oleyl | 10 | 1.77 | 33 | 14.5 |
| | | | | alcohol | | | | |
| | Ex. 12 | 1.5 | — | Octyl | 10 | 1.01 | 34 | 10.7 |
| | | | | dodecanol | | | | |
| | Ex. 13 | 1.5 | — | Stearyl | 10 | 1.83 | >45 | 6.0 |
| | | | | alcohol | | | | |
| Ex. | Ex. 14 | 1.5 | 5 | Isopropyl | 10 | 3.16 | 15 | 59.3 |
| | | | | myristate | | | | |
| | Ex. 15 | 1.5 | 5 | Oleyl | 10 | 3.62 | 21 | 59.1 |
| | | | | alcohol | | | | |
| | Ex. 16 | 1.5 | 5 | Octyl | 10 | 2.74 | 15 | 47.5 |
| | | | | dodecanol | | | | |
| | Ex. 17 | 1.5 | 5 | Stearyl | 10 | 4.32 | 27 | 36.1 |
| | | | | alcohol | | | | |

(Jₘₐₓ: Maximum skin permeation rate, Tₘₐₓ: Time to reach the maximum skin permeation rate)

Imidafenacin, sorbitan monolaurate, isopropyl myristate, and alcohols were comprised in an OH-group-containing acrylic pressure-sensitive adhesive base so as to achieve the blending ratios described in the above Table, and percutaneous absorption preparations were prepared.

For each of the percutaneous absorption preparations, skin permeability was measured using the skin of a hairless mouse as described above; regarding the pressure-sensitive adhesive compositions as comparative examples, i.e., that without sorbitan monolaurate, isopropyl myristate and alcohols (Example 1), and that with any one of them (Examples 3 and 10-13), a long time was required to reach the maximum skin permeation rate, and the value of the maximum skin permeation rate was low. In contrast, regarding the pressure-sensitive adhesive compositions as examples containing both sorbitan monolaurate and isopropyl myristate or alcohols (Examples 14-17), a skin permeation rate of greater than 2.0 μg/cm²/hr was measured at 15 hr after the application; namely, a high Jₘₐₓ value was obtained within a short period of time (Tₘₐₓ), and also a large cumulative amount of permeation exceeding 30 μg/cm² was obtained.

Thus, the percutaneous absorption preparations comprising sorbitan monolaurate and either isopropyl myristate or alcohols were demonstrated to have synergistically increased skin permeability of imidafenacin.

TABLE 3
[Evaluation of skin permeability of silodosin (sorbitan
monolaurate)]

| | | | | | Skin permeability in | | |
|---|---|---|---|---|---|---|---|
| | | | | | hairless mouse | | |
| | | | | | | | Cumulative |
| | | | | Sorbitan | | | amount of |
| | | Silodosin | Triacetin | monolaurate | Jₘₐₓ | Tₘₐₓ | permeation |
| | | (mass %) | (mass %) | (mass %) | (μg/cm²/hr) | (hr) | (μg/cm²) |
| Com. | Ex. 18 | 5 | — | — | 2.97 | 17 | 36.4 |
| Ex. | Ex. 19 | 5 | 9 | — | 7.12 | 21 | 75.1 |
| | Ex. 20 | 5 | — | 3 | 3.85 | 15 | 50.2 |
| Ex. | Ex. 21 | 5 | 9 | 3 | 14.71 | 13 | 186.0 |
| | Ex. 22 | 5 | 18 | 3 | 15.58 | 13 | 205.6 |
| | Ex. 23 | 5 | 9 | 5 | 12.71 | 13 | 168.0 |

(Jₘₐₓ: Maximum skin permeation rate, Tₘₐₓ: Time to reach the maximum skin permeation rate)

Silodosin, triacetin and sorbitan monolaurate were comprised in an OH-group-containing acrylic pressure-sensitive adhesive base so as to achieve the blending ratios described in the above Table, and percutaneous absorption preparations were prepared.

For each of the percutaneous absorption preparations, skin permeability was measured using the skin of a hairless mouse as described above; regarding the pressure-sensitive adhesive compositions as comparative examples, i.e., that without triacetin and sorbitan monolaurate (Example 18), and that with any one of them (Examples 19 and 20), a long time was required to reach the maximum skin permeation rate, and the value of the maximum skin permeation rate was low. In contrast, regarding the pressure-sensitive adhesive compositions as examples containing both triacetin and sorbitan monolaurate (Examples 21-23), a skin permeation rate of greater than 7.0 μg/cm²/hr was measured at 13 hr after the application; namely, a high Jₘₐₓ value was obtained within a short period of time (Tₘₐₓ), and also a large cumulative amount of permeation exceeding 100 μg/cm² was obtained.

Thus, the percutaneous absorption preparations comprising triacetin and sorbitan monolaurate were demonstrated to have synergistically increased skin permeability of silodosin.

TABLE 4
[Evaluation of skin permeability of silodosin (lauric acid
diethanolamide)]

| | | | | | Skin permeability in | | |
|---|---|---|---|---|---|---|---|
| | | | | | hairless mouse | | |
| | | | | Lauric | | | Cumulative |
| | | | | acid | | | amount of |
| | | Silodosin | Triacetin | diethanolamide | Jₘₐₓ | Tₘₐₓ | permeation |
| | | (mass %) | (mass %) | (mass %) | (μg/cm²/hr) | (hr) | (μg/cm²) |
| Com. | Ex. 18 | 5 | — | — | 2.97 | 17 | 36.4 |
| Ex. | Ex. 24 | 5 | 3 | — | 5.67 | >23 | 42.1 |
| | Ex. 25 | 5 | — | 3 | 4.46 | 11 | 80.3 |
| Ex. | Ex. 26 | 5 | 3 | 3 | 9.54 | 11 | 127.4 |
| | Ex. 27 | 5 | 6 | 3 | 10.79 | 11 | 141.0 |
| | Ex. 28 | 5 | 9 | 3 | 13.47 | 9 | 166.4 |
| | Ex. 29 | 5 | 9 | 1 | 9.42 | 13 | 135.1 |
| | Ex. 30 | 5 | 9 | 2 | 11.93 | 13 | 155.1 |
| | Ex. 31 | 3 | 9 | 3 | 11.12 | 11 | 142.0 |
| | Ex. 32 | 7 | 9 | 3 | 14.86 | 11 | 193.2 |
| | Ex. 33 | 9 | 9 | 3 | 15.4 | 13 | 210.1 |

(Jₘₐₓ: Maximum skin permeation rate, Tₘₐₓ: Time to reach the maximum skin permeation rate)

Silodosin, triacetin, and lauric acid diethanolamide were comprised in an OH-group-containing acrylic pressure-sensitive adhesive base so as to achieve the blending ratios described in the above Table, and percutaneous absorption preparations were prepared.

For each of the percutaneous absorption preparations, skin permeability was measured using the skin of a hairless mouse as described above; regarding the pressure-sensitive adhesive compositions as comparative examples, i.e., that without triacetin and lauric acid diethanolamide (Example 18), and that with any one of them (Examples 24 and 25), a long time was required to reach the maximum skin permeation rate, and the value of the maximum skin permeation rate was low. In contrast, regarding the pressure-sensitive adhesive compositions as examples containing both triacetin and lauric acid diethanolamide (Examples 26-33), a skin permeation rate of greater than 7.0 μg/cm²/hr was measured at 13 hr after the application; namely, a high Jₘₐₓ value was obtained within a short period of time (Tₘₐₓ), and also a large cumulative amount of permeation exceeding 100 μg/cm² was obtained. Thus, the percutaneous absorption preparations comprising triacetin and lauric acid diethanolamide were demonstrated to have synergistically increased skin permeability of silodosin.

TABLE 5
[Time-course change in permeability]

| | Ex. 3 | | Ex. 11 | | Ex. 4 | | Ex. 14 | | Ex. 15 | |
|---|---|---|---|---|---|---|---|---|---|---|
| Time | Flux | SD | Flux | SD | Flux | SD | Flux | SD | Flux | SD |
| (hr) | (μg/cm²/hr) | | | | | | | | | |
| 0 | 0 | 0 | 0 | 0 | 0 | 0 | 0 | 0 | 0 | 0 |
| 3 | 0.13 | 0.049 | 0.03 | 0.020 | 0.21 | 0.017 | 0.69 | 0.027 | 0.35 | 0.076 |
| 9 | 1.41 | 0.254 | 0.31 | 0.151 | 2.54 | 0.060 | 2.88 | 0.245 | 2.59 | 0.135 |
| 15 | 2.20 | 0.326 | 0.77 | 0.214 | 3.53 | 0.243 | 3.16 | 0.077 | 3.29 | 0.275 |
| 21 | 2.22 | 0.365 | 1.32 | 0.250 | 2.63 | 0.180 | 3.15 | 0.194 | 3.62 | 0.632 |
| 27 | 2.15 | 0.298 | 1.76 | 0.239 | 1.88 | 0.081 | 2.94 | 0.237 | 3.50 | 0.519 |
| 33 | 2.00 | 0.213 | 1.77 | 0.235 | 1.51 | 0.033 | 2.55 | 0.238 | 2.91 | 0.140 |
| 39 | 1.77 | 0.138 | 1.58 | 0.227 | 1.26 | 0.022 | 2.01 | 0.178 | 2.11 | 0.169 |
| 45 | 1.54 | 0.072 | 1.42 | 0.181 | 1.12 | 0.033 | 1.52 | 0.146 | 1.49 | 0.255 |

Time-course changes in the amount of permeation (Flux, μg/m²/hr) of the imidafenacin-containing (1.5 mass %) percutaneous absorption preparations comprising the following substances are shown in the above Table 5 and the graph in FIG. 1: Example 3 (sorbitan monolaurate 5 mass %), Example 11 (oleyl alcohol 10 mass %), Example 4 (sorbitan monolaurate 5 mass %+triacetin 10 mass %), Example 14 (sorbitan monolaurate 5 mass %+isopropyl myristate 10 mass %), and Example 15 (sorbitan monolaurate 5 mass %+oleyl alcohol 10 mass %).

When triacetin is used, Flux value tends to decrease rapidly after reaching the time to reach the maximum skin permeation rate (Tₘₐₓ); whereas when oleyl alcohol or isopropyl myristate is used, a high Flux value tends to be maintained even after reaching Tₘₐₓ. Thus, a percutaneous absorption promoting agent can be appropriately selected depending on the intended use (dose regimen and dosage).

### INDUSTRIAL APPLICABILITY

As described above, the percutaneous absorption preparation of the present invention enables to provide good skin permeability of drugs by comprising a percutaneous absorption promoting agent and fatty acid ester and/or fatty acid amide; and therefore, the preparation can be used with a drug having low skin permeability such as imidafenacin and silodosin, and therefore it is extremely useful for the treatment of urinary frequency, urinary incontinence and dysuria.

1. A percutaneous absorption preparation comprising a drug and one or more percutaneous absorption promoting agents selected from triacetin, isopropyl myristate, oleyl alcohol, octyldodecanol and stearyl alcohol for said drug, wherein the percutaneous absorption preparation further comprises a fatty acid ester and/or a fatty acid amide that further improve the function of the percutaneous absorption promoting agents.

2. The percutaneous absorption preparation according to claim 1, wherein the drug is imidafenacin and/or a salt thereof, or silodosin and/or a salt thereof.

3. The percutaneous absorption preparation according to claim 1, wherein the fatty acid ester is sorbitan monolaurate.

4. The percutaneous absorption preparation according to claim 1, wherein the fatty acid amide is lauric acid diethanolamide.

5. The percutaneous absorption preparation according to claim 1, which is a skin external patch that comprises a pressure-sensitive adhesive composition comprising:
(1) imidafenacin and/or a salt thereof, or silodosin and/or a salt thereof, as a drug,
(2) triacetin,
(3) sorbitan monolaurate and/or lauric acid diethanolamide,
(4) a pressure-sensitive adhesive base.

6. The percutaneous absorption preparation according to claim 1, which is a skin external patch that comprises a pressure-sensitive adhesive composition comprising:
(1) imidafenacin and/or a salt thereof as a drug,
(2) triacetin, isopropyl myristate, oleyl alcohol, octyldodecanol, or stearyl alcohol,
(3) sorbitan monolaurate,
(4) a pressure-sensitive adhesive base.

7. The percutaneous absorption preparation according to claim 6, wherein at 15 hr after its application, the skin permeation rate is greater than 2.0 μg/cm²/hr and the cumulative amount of permeation is greater than 30 μg/cm².

8. The percutaneous absorption preparation according to claim 5, wherein it comprises silodosin and/or a salt thereof as a drug, and at 13 hr after its application, the skin permeation rate is greater than 7.0 μg/cm²/hr and the cumulative amount of permeation is greater than 100 μg/cm².

9. The percutaneous absorption preparation according to claim 5, wherein the pressure-sensitive adhesive base is (meth)acrylic acid ester copolymer.

10. The percutaneous absorption preparation according to claim 5, which is a skin external patch having a structure in which the pressure-sensitive adhesive composition is laminated on a backing and covered with a liner.

11. A method for producing a percutaneous absorption preparation comprising a drug and one or more percutaneous absorption promoting agents selected from triacetin, isopropyl myristate, oleyl alcohol, octyldodecanol and stearyl alcohol for said drug, wherein the percutaneous absorption preparation has increased skin permeability by means of further comprising a fatty acid ester and/or a fatty acid amide that further improve the function of said percutaneous absorption promoting agents.

12. The percutaneous absorption preparation according to any one of claim 6, which is a skin external patch having a structure in which the pressure-sensitive adhesive composition is laminated on a support and covered with a liner.

13. The percutaneous absorption preparation according to any one of claim 7, which is a skin external patch having a structure in which the pressure-sensitive adhesive composition is laminated on a support and covered with a liner.

14. The percutaneous absorption preparation according to any one of claim 8, which is a skin external patch having a structure in which the pressure-sensitive adhesive composition is laminated on a support and covered with a liner.

15. The percutaneous absorption preparation according to any one of claim 9, which is a skin external patch having a structure in which the pressure-sensitive adhesive composition is laminated on a support and covered with a liner.
